(19) United States
(12) Patent Application Publication
(10) Pub. No.: US 2009/0035334 A1
(43) Pub. Date: Feb. 5, 2009

(54) METHOD FOR PREPARING POLYMETHYLMETHACRYLATE SPHERICAL BEAD

(75) Inventors: Sun-jae Kim, Kyung-ki-do (KR); Hyoung-whee Kim, Kyung-ki-do (KR); Suk-woo Shin, Kyung-ki-do (KR); Han-song Chu, Kyung-ki-do (KR)

Correspondence Address: CANTOR COLBURN, LLP, 20 Church Street, 22nd Floor, Hartford, CT, 06103, US

(73) Assignee: SUNJIN CHEMICAL CO., LTD., Ansan city (KR)

(21) Appl. No.: 11/817,633

(22) PCT Filed: Aug. 26, 2005
(86) PCT No.: PCT/KR2005/002822

§ 371 Date: Aug. 13, 2008

(30) Foreign Application Priority Data
Mar. 3, 2005 (KR) 10-2005-0017787

(51) Int. Cl. A61K 8/02; C08F 2/00

(52) U.S. Cl. 424/401; 526/77

(57) ABSTRACT

This invention provides a method for preparing a polymethylmethacrylate spherical bead and in particular, it provides a method for preparing a polymethylmethacrylate spherical bead characterized in that it is prepared by preparing polymethylmethacrylate by polymerizing methyl-methacrylate monomers, evaporating remaining methylmethacrylate monomers by raising temperature up to at least 100° C. after the polymerization reaction, and washing the polymethylmethacrylate with a mixed solvent of ethanol and water and vacuum drying them. The polymethylmethacrylate spherical beads in accordance with the invention not only can provide pure polymethylmethacrylate in its odorless condition by completely eliminating the remaining methylmethacrylate monomers that generate off-odor but also show no yellowing and at the same time, they are suitable for cosmetic materials or industrial materials because they are harmless to human and have no off-odor

### TECHNICAL FIELD

The present invention relates to a method for preparing a polymethylmethacrylate spherical bead and more particularly, it relates to a method for preparing a polymethylmethacrylate spherical bead suitable for use as cosmetic materials or industrial materials, which not only can provide pure polymethylmethacrylate in its odorless condition by completely eliminating remaining methylmethacrylate monomers that generate off-odor but also show no yellowing and at the same time, which is harmless to human and does not have off-odor.

### BACKGROUND ART

In general, polymethylmethacrylate has been mostly used as a powder in functional cosmetics, UV block creams, materials for feeling improvement in colour cosmetics, etc.

This polymethylmethacrylate is prepared by mixing methylmethacrylate monomers, a cross linking agents and an initiator and polymerizing them, then dehydrating and drying them and comminuting them.

However, as polymethylmethacrylate prepared by such a method contains methylmethacrylate in a large amount, it can cause irritation such as allergy when contacted with skin, in worse case, it may cause rash, burn, etc. and if it is inhaled or ingested, it may cause nausea, coma conditions, etc.

Also, methylmethacrylate generates off-odor and accordingly, when polymethylmethacrylate is used alone or it is used in a mixture with other materials, it can give rise to off-odor.

Therefore, study about methods for the preparation of polymethylmethacrylate, which is capable of preventing the generation of off-odor and harmless to human by completely eliminating methylmethacrylate monomers from polymethylmethacrylate that is a cosmetic material in direct contact with skin is greatly needed.

### DISCLOSURE OF INVENTION

### Technical Problem

To solve the problems as described in the above, it is an object of the present invention to provide a method for preparing a polymethylmethacrylate spherical bead capable of providing pure polymethylmethacrylate in its odorless condition by completely eliminating remaining methylmethacrylate monomers that generate off-odor.

Also, it is another object of the invention to provide a method for preparing pure polymethylmethacrylate spherical bead in its odorless condition that is harmless to human by completely eliminating methylmethacrylate that is harmful to human and generates off-odor from polymethylmethacrylate that is used as a cosmetic material, a polymethylmethacrylate spherical bead prepared by the same method, and a cosmetic composition comprising the polymethylmethacrylate spherical bead.

### Technical Solution

To achieve the aforementioned objects, the present invention provides a method for preparing a polymethylmethacrylate spherical bead comprising the steps of:
a) preparing polymethylmethacrylate by polymerizing methylmethacrylate monomers;
b) evaporating remaining methylmethacrylate monomers by raising temperature up to at least 100° C. after the polymerization reaction of a); and
c) washing the polymethylmethacrylate of b) with a mixed solvent of ethanol and water and vacuum drying them.

Also, the invention provides a polymethylmethacrylate spherical bead characterized in that it is prepared by the above method.

Also, the invention provides a cosmetic composition characterized in that the polymethylmethacrylate spherical bead is applied thereto.

### Advantageous Effects

The polymethylmethacrylate of the invention prepared by the above method not only can completely eliminate remaining methylmethacrylate monomers that generate off-odor but also shows no yellowing and at the same time, it can provide a pure polymethylmethacrylate spherical bead in its odorless condition that is harmless to human by completely eliminating methylmethacrylate that is harmful to human and generates off-odor from polymethylmethacrylate spherical beads that are used as cosmetic materials.

### BRIEF DESCRIPTION OF THE DRAWINGS

FIG. 1 is a photograph of a polymethylmethacrylate spherical bead prepared by ethanol washing and vacuum dry in accordance with one embodiment of the present invention.

FIG. 2 is a photograph of a polymethylmethacrylate spherical bead prepared by heated air drying without ethanol washing in accordance with Comparative Example 3.

### MODE FOR THE INVENTION

The present invention is further described in detail.

The inventors found that remaining methylmethacrylate monomers could be completely eliminated through vacuum dry method by reducing residual methylmethacrylate monomers as much as possible prior to dry step and has completed the present invention on the basis of that finding.

The polymethylmethacrylate spherical bead of the invention is characterized in that it is prepared by the step of preparing polymethylmethacrylate by polymerizing methylmethacrylate monomers, the step of evaporating remaining methylmethacrylate monomers by raising temperature up to at least 100° C. after the polymerization reaction, and the step of washing the polymethylmethacrylate with a mixed solvent of ethanol and water and vacuum drying them.

For the methylmethacrylate monomers of step a) in the invention, methylmethacrylate monomers can be used alone, or methylmethacrylate monomers can be used in a mixture with a small amount of acrylate monomers.

For the mixable acrylate monomers, preferably, alkyl acrylate monomers having 1 to 8 carbon atoms can be used and more particularly, methyl acrylate, ethyl acrylate, butyl acrylate, or 2-ethyl hexyl acrylate can be used.

The acrylate monomers are mixed preferably in an amount of 0 to 20 parts by weight with regard to 100 parts by weight of the methylmethacrylate monomers.

The polymethylmethacrylate of step a) can be prepared by polymerizing the methylmethacrylate monomers after adding a crosslinking agent and initiator thereto.

For the crosslinking agent, ordinary crosslinking agents that are used to prepare polymethylmethacrylate can be used and for the initiator, ordinary initiators that are used to prepare polymethylmethacrylate can be used and particularly, azo-based initiators such as 2,2′-azobis 2′4-dimethyl-valeronitrile can be preferably used.

After the mixing of the methylmethacrylate monomers, crosslinking agent and initiator, polymethylmethacrylate can be prepared by bulk polymerization, suspension polymerization, emulsion polymerization, solution polymerization, etc. and particularly, in this invention, emulsion polymerization is preferable.

The prepared polymethylmethacrylate is treated to evaporate remaining methylmethacrylate monomers by raising the reaction temperature up to at least 100° C. Preferably, the evaporated methylmethacrylate monomers are all treated with a scrubber so as to prevent air pollution.

If the reaction temperature is lower than 100° C., the elimination efficiency of the residual methylmethacrylate monomers remaining in the polymethylmethacrylate is decreased.

After the remaining methylmethacrylate monomers are evaporated, the polymethylmethacrylate is washed with a mixed solvent of ethanol and water and then vacuum dried.

The washing step reduces the amount of the methylmethacrylate monomers remaining in the polymethylmethacrylate and at the same time, it eliminates impurities such as emulsifying agents remaining after the polymerization reaction and thus can prevent discoloring due to impurities even when temperature is increased during dry step.

The mixed solvent used in the washing step uses ethanol and water preferably in a ratio of 1:1 to 1:5 and the polymethylmethacrylate powder is washed with the mixed solvent preferably in a ratio of 1:2 to 1:5.

The washing is carried out preferably for 3 to 5 hours and more preferably for 4 hours.

After washing, the reaction mixture is dehydrated using a centrifuge for 3 to 4 hours and then subject to vacuum dry step. The vacuum dry can be carried out with variation in pressure, temperature and time, if necessary and particularly, it is carried out preferably at the pressure of 1/100 to 1/10000 torr at the temperature of 85 to 95° C. for 48 to 96 hours. If the pressure deviates from 1/100 to 1/10000 torr ranges, remaining methylmethacrylate may not be completely eliminated and if the temperature exceeds 95° C., the feel of the polymethylmethacrylate spherical bead that is finally obtained is not good.

Also, the present invention provides a polymethylmethacrylate spherical bead prepared by the above method. The polymethylmethacrylate spherical bead of the invention not only can completely eliminate remaining methylmethacrylate monomers which generate off-odor but also shows no yellowing and at the same time, it is the pure polymethylmethacrylate in its odorless condition that is harmless to human by the complete elimination of methylmethacrylate that is harmful to human and generates off-odor.

Also, this invention provides a cosmetic composition comprising a polymethylmethacrylate spherical bead prepared by the above method and the polymethylmethacrylate spherical bead is applicable to ordinary cosmetic compositions such as colour cosmetics, UV block creams and functional cosmetics.

For better understanding of the present invention, preferred examples follow. The following examples are intended to illustrate the invention more fully without limiting the scope of the invention.

### EXAMPLES

### Example 1

100 parts by weight of methylmethacrylate monomers, 10 parts by weight of 2-propenic acid,2-methyl-1,2-ethanediyl ester as a crosslinking agent, 0.05 parts by weight of 2,2′-azobis 2,4′-dimethyl-valeronitrile as an initiator, and 0.2 parts by weight of polyvinyl alcohol as an emulsifying agent were mixed and emulsion polymerized to prepare polymethylmethacrylate.

After the polymerization was complete, the reaction temperature was raised to 100° C. whereby methylmethacrylate monomers remaining in the polymethylmethacrylate was evaporated. The whole amount of the evaporated methylmethacrylate monomers was treated with a scrubber.

Then, the polymethylmethacrylate was washed with a mixed solvent of ethanol and water in a ration 1:2 for 4 hours and dehydrated using a centrifuge for 3 to 4 hours and then, it was vacuum dried at the pressure of 1/200 torr at the temperature of 90° C. for 72 hours to prepare a final polymethylmethacrylate spherical bead powder.

### Example 2

With the exception that a mixture of 100 parts by weight of methylmethacrylate monomers and 10 parts by weight of methylacrylate was used in Example 1, a final polymethylmethacrylate spherical bead powder was prepared by the same method as Example 1.

### Example 3

With the exception that the vacuum dry was carried out at the pressure of 1/200 torr at the temperature of 90° C. for 48 hours in Example 1, a final polymethylmethacrylate spherical bead powder was prepared by the same method as Example 1.

### Example 4

With the exception that the vacuum dry was carried out at the pressure of 1/200 torr at the temperature of 85° C. for 72 hours in Example 1, a final polymethylmethacrylate spherical bead powder was prepared by the same method as Example 1.

### Example 5

With the exception that the vacuum dry was carried out at the pressure of 1/100 torr at the temperature of 90° C. for 72 hours in Example 1, a final polymethylmethacrylate spherical bead powder was prepared by the same method as Example 1.

### Comparative Example 1

A final polymethylmethacrylate spherical bead powder was prepared by heated air drying of the polymethylmethacrylate prepared in Example 1.

### Comparative Example 2

A final polymethylmethacrylate spherical bead powder was prepared by air flow drying of the polymethylmethacrylate prepared in Example 1.

### Comparative Example 3

A final polymethylmethacrylate spherical bead powder was prepared by vacuum drying the polymethylmethacrylate prepared in Example 1 at the pressure of 1/200 torr at the temperature of 90° C. for 4 hours without raise in reaction temperature and washing step.

The final polymethylmethacrylate powders prepared in Example 1 to 5 and Comparative Examples 1 to 3 were mixed with ethanol so that the ratio of the ethanol and the powder was 9:1 and stirred at 25° C. for 20 min. Then, the amount of the methylmethacrylate monomers remaining in the final polymethylmethacrylate spherical bead powder was determined by measuring GC using the solution from which the powder was filtrated off and the results are shown in Table 1 below.

TABLE 1

| | | | | | | Comparative | | |
|---|---|---|---|---|---|---|---|---|
| | Examples | | | | | Examples | | |
| | 1 | 2 | 3 | 4 | 5 | 1 | 2 | 3 |
| Amount of | 0 | 0 | 0 | 0 | 0 | 326 | 586 | 47 |
| Methylmethacrylate | | | | | | | | |
| Monomers (ppm) | | | | | | | | |

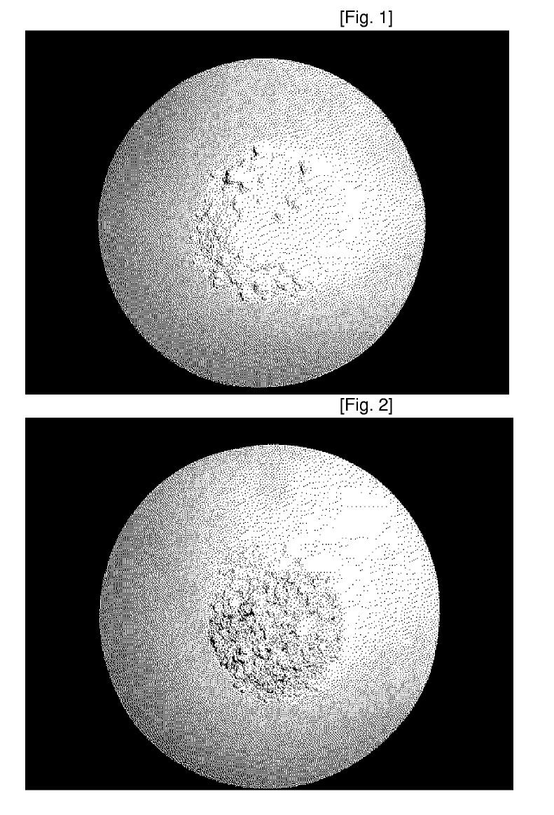

From Table 1 above, it can be seen that unlike Comparative Examples 1 to 3, no methylmethacrylate monomers were detected within the final polymethylmethacrylate spherical beads in Examples 1 to 5 prepared in accordance with the present invention, and while the polymethylmethacrylate spherical bead prepared in Example 1 of the invention where the ethanol washing and vacuum dry step were performed showed no yellowing (FIG. 1), the polymethylmethacrylate prepared by Comparative Example 3 showed yellowing phenomena as shown in FIG. 2.

Also, it can be seen that as the polymethylmethacrylate prepared in accordance with the invention completely eliminated methylmethacrylate monomers that are harmful to human and have off-odor, it is suitable for use as a cosmetic material.

### INDUSTRIAL APPLICABILITY

The polymethylmethacrylate of invention prepared in the above not only can completely eliminate remaining methylmethacrylate monomers that generate off-odor but also shows no yellowing and at the same time, it can provide a pure polymethylmethacrylate spherical bead in its odorless condition that is harmless to human by completely eliminating methylmethacrylate that is harmful to human and generates off-odor from the polymethylmethacrylate spherical beads used as cosmetic materials.

1. A method for preparing polymethylmethacrylate spherical bead comprising the steps of:
a) preparing polymethylmethacrylate by polymerizing methylmethacrylate monomers;
b) evaporating remaining methylmethacrylate monomers by raising temperature up to at least 100° C. after the polymerization reaction of a); and
c) washing the polymethylmethacrylate of b) with a mixed solvent of ethanol and water and vacuum drying them.

2. The method for preparing the polymethylmethacrylate spherical bead of claim 1 wherein the methylmethacrylate monomers of step a) is used alone or in a mixture with acrylate monomers.

3. The method for preparing the polymethylmethacrylate spherical bead of claim 2 wherein the acrylate monomers are alkyl acrylate monomers having 1 to 8 carbon atoms selected from the group consisting of methyl acrylate, ethyl acrylate, butyl acrylate, 2-ethyl hexyl acrylate and mixture thereof.

4. The method for preparing the polymethylmethacrylate spherical bead of claim 2 wherein the acrylate monomers are mixed with the methylmethacrylate monomers in a ratio of 0 to 20 parts by weight with regard to 100 parts by weight of the methylmethacrylate monomers.

5. The method for preparing the polymethylmethacrylate spherical bead of claim 1 wherein the ethanol and water were mixed in a ratio of 1:1 to 1:5 in the mixed solvent of step c).

6. The method for preparing the polymethylmethacrylate spherical bead of claim 1 wherein the vacuum dry of step c) is carried out at the pressure of 1/100 to 1/10000 torr at the temperature of 85 to 95° C. for 48 to 96 hours.

7. A polymethylmethacrylate spherical bead prepared by the method described in claim 1.

8. A cosmetic comprising the polymethylmethacrylate spherical bead described in claim 7.

9. A polymethylmethacrylate spherical bead prepared by the method described in claim 2.

10. A cosmetic comprising the polymethylmethacrylate spherical bead described in claim 9.

11. A polymethylmethacrylate spherical bead prepared by the method described in claim 3.

12. A cosmetic comprising the polymethylmethacrylate spherical bead described in claim 11.

13. A polymethylmethacrylate spherical bead prepared by the method described in claim 4.

14. A cosmetic comprising the polymethylmethacrylate spherical bead described in claim 13.

15. A polymethylmethacrylate spherical bead prepared by the method described in claim 5.

16. A cosmetic comprising the polymethylmethacrylate spherical bead described in claim 15.

17. A polymethylmethacrylate spherical bead prepared by the method described in claim 6.

18. A cosmetic comprising the polymethylmethacrylate spherical bead described in claim 17.
